(19) United States
(12) Patent Application Publication
(10) Pub. No.: US 2026/0198914 A1
(43) Pub. Date: Jul. 16, 2026

(54) POROUS SUTURE AND METHOD OF USE

(71) Applicant: Deep Blue Medical Advances, Inc., Durham, NC (US)

(72) Inventors: Howard Levinson, Chapel Hill, NC (US); David Ruppert, Chapel Hill, NC (US); William A. Perry, San Francisco, CA (US)

(73) Assignee: Deep Blue Medical Advances, Inc., Durham, NC (US)

(21) Appl. No.: 19/560,801

(22) Filed: Mar. 9, 2026

Related U.S. Application Data
(63) Continuation of application No. 18/435,674, filed on Feb. 7, 2024, now abandoned.
(60) Provisional application No. 63/444,459, filed on Feb. 9, 2023.

(51) Int. Cl. A61B 17/04

(52) U.S. Cl. CPC A61B 17/04

(57) ABSTRACT

A porous suture for tissue repair includes a planar body has a length between a first end and a second end and a width between a first lateral side and a second lateral side, the planar body being formed of a porous material comprising a plurality of pores along the length of the planar body between the first end and the second end. The planar body is configured to substantially maintain the width despite an increase in a lengthwise force on the planar body.

### CROSS REFERENCE TO RELATED APPLICATION

This application claims priority to U.S. application Ser. No. 18/435,674, filed Feb. 7, 2024 and U.S. Provisional Application No. 63/444,459, filed Feb. 9, 2023, all of which applications are incorporated herein by reference in their entirety.

### FIELD

The present disclosure relates to sutures for implantation into tissue of a human or other animal and suturing methods for implantation and fixation of sutures to tissue.

### BACKGROUND

Sutures are used to make stitches for holding tissues together, or otherwise positioning or supporting tissue for healing and/or regrowth, such as to close a wound, repair a tissue defect, or for any other type of tissue repair. Sutures are used in surgical procedures for wound closure, to close the skin in plastic surgery, to secure damaged or severed tendons, to repair muscles or other internal tissues, or to affix an implant to tissue. Sutures can be introduced into the tissue by a fixation device, which may include an introducing device such as a needle or other insertion device attached to one or multiple ends of the suture. Generally, the suture needle or device is intended to penetrate and pass through the tissue, pulling the suture through the tissue. For example, the needle may be passed through opposing faces of tissue, which are then moved together as the suture is pulled taut. The suture may be tied, knotted, or fixated in some way to secure it. Once a stitch or set of stitches is complete, the suture is cut or the needle is otherwise removed from the end of the suture.

### SUMMARY

This Summary is provided to introduce a selection of concepts that are further described below in the Detailed Description. This Summary is not intended to identify key or essential features of the claimed subject matter, nor is it intended to be used as an aid in limiting the scope of the claimed subject matter.

In one aspect of the disclosure, a porous suture for tissue repair includes a planar body having a length between a first end and a second end and a width between a first lateral side and a second lateral side, the planar body being formed of a porous material comprising a plurality of pores along the length of the planar body between the first end and the second end. The planar body is configured to substantially maintain the width despite an increase in a lengthwise force on the planar body.

In one embodiment, the width of the suture decreases by no more than 40% when the lengthwise force increases up to 16N compared to the width under no lengthwise force.

In another embodiment, the width of the suture decreases by no more than 40% when the lengthwise force increases up to 32N compared to the width under no lengthwise force.

In another embodiment, the width of the suture decreases by no more than 40% when the lengthwise force increases up to 50N compared to the width under no lengthwise force.

In another embodiment, the width of the suture decreases by no more than 30% when the lengthwise force increases up to 16N compared to the width under no lengthwise force.

In another embodiment, the width of the suture decreases by no more than 20% when the lengthwise force increases up to 16N compared to the width under no lengthwise force.

In another embodiment, the width of the suture decreases by no more than 10% when the lengthwise force increases up to 16N compared to the width under no lengthwise force.

In another embodiment, wherein the planar body is configured to remain flat along its width despite the increase in lengthwise force.

In another embodiment, wherein the planar body comprises a plurality of strands forming chain stitches along the length.

In another embodiment, the planar body comprises a single ply of the porous material.

In another embodiment, the porous material is a mesh.

In another embodiment, the porous material is a mesh and further comprises a set of cross-connecting strands connecting chain stitches to form the mesh.

In another embodiment, the porous material is a mesh wherein the mesh forms the plurality of pores, and wherein a pore size of the plurality of pores is consistent along the length of the planar body.

In another embodiment, the porous material is a mesh wherein the mesh forms the plurality of pores, and wherein a pore size of the plurality of pores is larger near the first end and/or the second end than in a middle portion of the planar body.

In another embodiment, the porous material is a mesh wherein the mesh forms the plurality of pores, and wherein a pore size of the plurality of pores is larger near a longitudinal centerline of the planar body than at the first lateral side or the second lateral side.

In another embodiment, the porous material is a continuous sheet, wherein the plurality of pores are formed through the continuous sheet.

In another embodiment, the plurality of pores are aligned along a longitudinal centerline of the planar body.

In another embodiment, at least a portion of the plurality of pores is sized to accommodate the porous suture being passed there through such that the porous suture is configured to be passed through itself.

In another embodiment, at least a portion of the plurality of pores is sized such that the porous suture is configured to be passed through itself to create a self-locking stitch.

In another embodiment, each of the plurality of pores is sized to accommodate the porous suture being passed there through such that the porous suture is configured to be passed through itself.

In another embodiment, the plurality of pores are uniformly spaced along the length of the planar body between the first end and the second end.

In another embodiment, the width of the porous suture is at least 4 mm.

In another embodiment, the porous suture is configured such that a ratio of the width to the length of the planar body does not substantially change when the porous suture is under high tension compared to the ratio under no lengthwise force.

In another embodiment, the ratio of the width to the length of the planar body is at least 1:10 when the porous suture is under no lengthwise force and under high tension.

In another embodiment, the ratio of the width to the length of the planar body is at least 1:20 when the porous suture is under no lengthwise force and under high tension.

In another embodiment, the porous suture comprises a first surgical needle fixed to the first end of the planar body and a second surgical needle fixed to the second end of the planar body.

In another aspect of the present disclosure, a method of repairing tissue with a porous suture, the porous suture is provided, the porous suture comprising a planar body having a length between a first end and a second end and a width between a first lateral side and a second lateral side, with a plurality of pores formed along the length of the planar body between the first end and the second end. The method includes passing the first end of the porous suture through tissue on each of a first side of a tissue repair site and a second side of the tissue repair site to create at least one stitch, and passing the suture through itself to create a self-locking stitch to create a finishing anchor at the first end of the porous suture.

In one embodiment, the method includes passing the first end of the porous suture through tissue on each of the first side and the second side of the tissue repair site multiple times to create a series of stitches to close a wound, wherein the self-locking stitch follows the series of stitches.

### BRIEF DESCRIPTION OF THE DRAWINGS

The present disclosure includes the following Figures.

FIGS. 1A-1C illustrates an embodiment of a porous suture with a curved fixation device and a straight fixation device.

FIGS. 2A-C illustrates another embodiment of a porous suture configured as a mesh suture.

FIGS. 3A-3D illustrate a method for anchoring a porous suture to a target surface with a clinch knot.

FIGS. 4A-4D illustrate a method for anchoring a porous suture to a target surface with a self-locking backstitch.

FIG. 5A-5D illustrate a method for anchoring a porous suture to a target surface with another self-locking weave pattern.

FIG. 6A-6C illustrate embodiments of a mesh suture including at least one barbed filament.

### DETAILED DESCRIPTION

In the present description, certain terms have been used for brevity, clarity and understanding. No unnecessary limitations are to be inferred therefrom beyond the requirement of the prior art because such terms are used for descriptive purposes only and are intended to be broadly construed.

The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Through experience and research in the relevant field, the present inventors have recognized several problems relating to solid sutures. After a suture is implanted in tissue, the tissue will begin to heal, and scar tissue forms around the suture. This creates a tube-like structure of scar tissue surrounding the suture with a small surface area. Available solid or other tubular sutures have generally smooth outer surfaces that do not adhere to or engage with scar tissue. As such, solid sutures can slide within the surrounding scar tissue with little resistance. As such, existing sutures can be easily removed and are prone to cutting tissue when used in high-tension applications. When a thin solid suture is acted upon by internal or external forces, it may dig into and damage the surrounding tissue. This “cheese wiring” effect can cause the suture to cut through and pull out of the tissue to which the suture is anchored.

In view of forgoing problems and challenges in the relevant art, the present inventors developed a novel porous suture that is configured for improved performance in high-tension applications. Embodiments of the porous suture disclosed herein include a generally planar body with a plurality of pores formed through the planar body. As the tissue heals around the porous suture, new scar tissue growth extends into the pores. This in-growth interlinks the porous suture with the surrounding tissue and inhibits sliding movement of the porous suture relative to the tissue. Furthermore, the in-growth of the tissue in combination with the wide body of the novel porous suture distributes the forces acting on the tissue to prevent the porous suture from cutting through the surrounding tissue when placed under tension.

Additionally, the porous suture is constructed to enable it to be passed through itself at the pore location for improved fixation. The pores of the porous suture are configured to enable the suture and/or a fixation device, such as a needle, to pass through the suture body to form a locking backstitch or other anchoring stitch. Passing the suture through itself provides a flatter and less bulky knot that stays relatively flush at the anchoring location. This flatter profile knot is less palpable, yielding less patient discomfort, and is less likely to cause tissue erosion or infection at the anchor location.

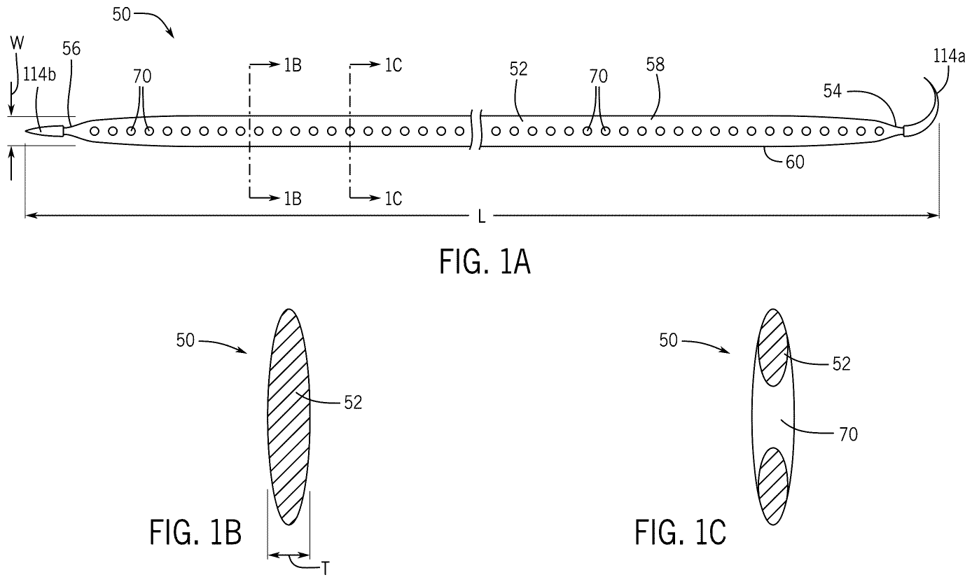

FIGS. 1A-1C illustrates an embodiment of a novel porous suture 50 configured for biological applications, such as for wound closure and/or high-tension tissue repair. FIG. 1A shows a planar view of a porous suture 50 and FIGS. 1B and 1C show cross-sections taken along the lines noted in FIG. 1A. The suture 50 has a flexible, generally planar body 52 that extends longitudinally from a first end 54 to an opposite second end 56 and laterally between opposing first and second lateral sides 58, 60. In some embodiments, the porous suture may have more than two ends, such as a length of porous suture that splits into multiple lengths of porous suture at some point along its length L and thus provides one side with multiple ends. The body 52 of the porous suture 50 may be formed from a resiliently deformable material configured to maintain a planar shape while the porous suture 50 is under tension. This may be useful, for example, to maximize the surface area of the porous suture 50 on the target surface, thereby decreasing stress and distributing force in the target surface and reducing the risk of anchor point failure. The planar body 52 may also provide a thin cross-sectional area that can be easily penetrated by the first or second end 54, 56 of the porous suture 50 for fixation to the target surface.

The body 52 of the porous suture 50 is constructed from any biocompatible material and may be configured to be permanent, removed, or degradable. For example, the porous suture 50 may include one or more materials or filaments that may comprise a biocompatible metal of a permanent nature (e.g., stainless steel, titanium, etc.) or a degradable nature (e.g., magnesium alloy, etc.); a biocompatible synthetic polymer of a permanent nature (e.g., polypropylene, polyester, etc.) or a degradable nature (e.g., polylactic acid, polypropylene fumarate, polylactic-co-glycolic acid, etc.); and/or a collagen-based material (e.g., allograft, xenograft, etc.). For example, the body 52 of the porous suture 50 may be formed of multiple filaments or textile strands, which may include a mix of any of the above-listed materials, and may be a knitted, woven, sewn, spun, and/or braided fabric body 52. Alternatively or additionally, the porous suture may be formed with drug-eluding materials containing therapeutics or materials treated with therapeutics. Alternatively or additionally, the porous suture may be formed by cutting (e.g. laser, die, etc.) and/or printing (e.g. FDM, SLA. DLP, SLM, etc.)

Embodiments of a suture 50 may include at least one fixation device 114 that is attached to the first end 54 and/or the second end 56 of the body 52, or at each of multiple ends. The fixation devices 114a, 114b may be any element or series of elements that enable fixation of the porous suture 50 to the target surface. Exemplary fixation devices 114 include, but are not limited to, surgical needles or other rigid or semi-rigid bodies formed at one or both ends of the suture, staples, tacks, screws, laser-assisted tissue welding, fibrin sealant, glue, salute “Q” ring, Mitek anchors, and/or other sutures. Each fixation device 114 may be permanently, degradably, or removably attached to a location of the suture. Where the fixation device 114 is configured for passing the suture through the tissue, such as the needles 114a, 114b shown in FIG. 1, it may be permanently or removably attached to the first and/or second end 54, 56 of the porous suture 50. Alternatively or additionally, the fixation device 114 may be permanently, degradably, or removably attached to another portion of the body 52. Moreover, the fixation device 114 may be an element that is permanently or transiently implanted in the patient, or that is removed from the porous suture 50 once it is implanted in a patient.

The fixation devices 114a, 114b shown in FIG. 1 are surgical needles configured to assist in affixing the porous suture 50 into the target surface—i.e., to allow the user to pass the porous suture 50 through the target surface and/or to pass the suture through itself or to otherwise form a stitch to secure it to the tissue, as described in more detail below. In particular, the porous suture 50 of FIG. 1 includes a first fixation device 114a configured as a curved needle attached to the first end 54 of the body 52 and a second fixation device 114b configured as a straight needle attached to the second end 56. In other embodiments, the needle may be an “s” shape or other multi-directional shape configured to forge a predefined path through tissue. The needles 114a, 114b may be formed of metal or of any rigid material suitable for penetrating tissue. Alternatively, the fixation device may be a flexible shaft containing the end of the porous suture. One or both ends may have fixations devices of either the same type or different. Inclusion of multiple fixation devices may be useful, for example, so that a surgeon may work in multiple directions and select a preferred needle type (i.e., leading with the first end 54 or the second end 56). Once the porous suture 50 has been secured in the target surface, the body 52 may be cut at the first end 54 and/or the second end 56 to remove the fixation device(s) 114a, 114b. Some embodiments, however, may be differently configured. For example, some embodiments may only include one fixation device attached to one end of a suture, or alternatively at a location other than the end. In other embodiments, elements may be formed at the ends of the porous suture 50, examples of which are described in more detail below.

With continued reference to FIGS. 1A-1C, the porous suture 50 includes a plurality of pores 70 formed along the length L of the body 52 between the first end 54 and the second end 54 thereof. As will be described in further detail below, each of the pores 70 is configured to allow the porous suture 50 to be passed through its body 52 via said pore 70 during attachment of the porous suture 50 to a target surface. In the illustrated embodiments, the pores 70 are uniformly spaced along the length L of the body 52 between the first and second ends 54, 56. In the embodiment of FIG. 1, the pores 70 are aligned along a longitudinal centerline of the planar body. Other embodiments, however, may be configured with a non-uniform arrangement of pores 70 which may be located along the centerline and/or elsewhere across the width W and length L of the body 52. As will be discussed in further detail below, the size of the pores 70, the shape of the pores 70, the spacing between pores 70, and/or the locations of the pores 70 in the suture body 52 may be different than those of the porous suture 50 of FIG. 1.

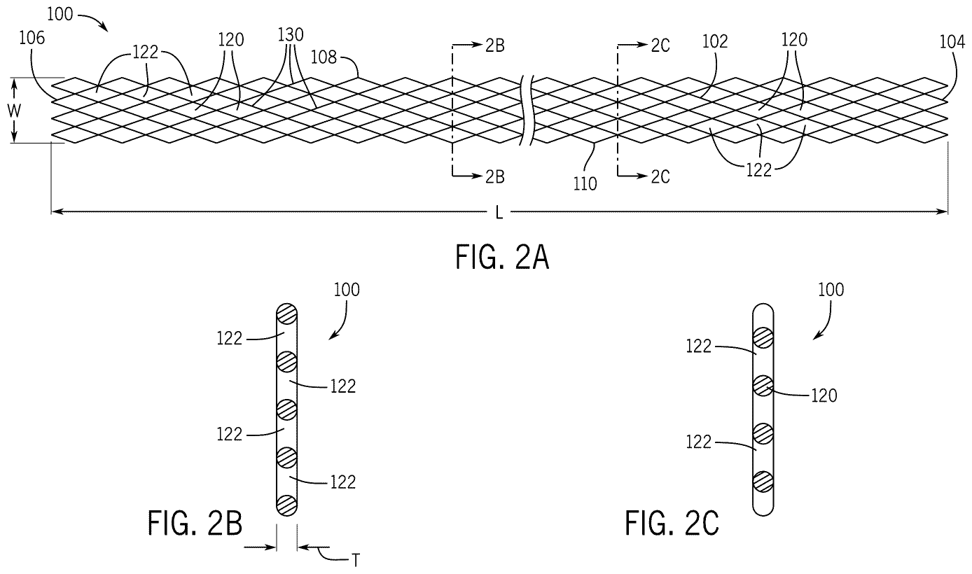

In the embodiment of FIGS. 1A-1C, the body 52 of the porous suture 50 is generally a continuous sheet or a relatively dense material with the pores 120 being holes formed through the sheet body 52. Some embodiments, however, may be differently configured. For example, referring to FIGS. 2A-2C, an embodiment of a porous suture 100 having a mesh suture body 102 is illustrated. Similarly to the embodiment of FIG. 1, the body 102 of the porous mesh suture 100 extends longitudinally from a first end 104 to a second end 106 and laterally between opposing first and second lateral sides 108, 110. Although not shown in FIGS. 2A-2C, embodiments of the mesh suture 100 may include a fixation device fixed or removably attached to the first end 104 and/or the second end 106 of the body 102, similar to that shown on the porous suture 50 of FIGS. 1A-1C and described above.

The body 52, 102 of the porous suture 50, 100 of FIGS. 1 and 2 is generally flat, such as having a substantially larger width W than thickness depth (and wherein the length L is substantially larger than the width W). The width W is substantially wider than that of a standard suture, such as a standard suture having a typical width in the range of 0.1 mm-0.7 mm, which provides the adhesion and tension-bearing benefits described herein. For example, the width may be at least 2 mm, and for many tissue repair applications may preferably be greater than 3 mm, or in some implementations at least 4 mm, or in some implementations at least 5 mm. Exemplary lengths L, widths W, and thicknesses, and desirable proportions for such dimensions, are described below. The generally flat body 52, 102 may be formed of a single-ply material or fabric. Alternatively, it may be formed of a multi-ply fabric where the pores 70, 120 provide a passage or path through all of the plies, or layers. As described in more detail below, the material of the body 52, 102 may be configured to maintain its width W when under lengthwise tension, or at least a substantial portion of the original width W that it has when not under tension. This enables the porous suture 50, 100, once implanted, to distribute the load across a wider area of tissue compared to prior art sutures, and also enables tissue ingrowth and adhesion to provide superior performance for high-tension applications (such as tendon repairs, muscle repairs, ligament repairs, fascia repairs, and breast tissue repairs).

The body 102 of the mesh porous suture 100 may be an arrangement of biocompatible textile strands 130 that are knitted, woven, sewn, braided, and/or otherwise linked to form a continuous, flexible material. The body 52 of the continuous sheet suture 50 may also be formed of textile strands arranged in a denser formation than that of the mesh suture body 102. Each of the textile strands 130 that form the body 52, 102 may be, for example, monofilaments, braided filaments, a combination of monofilament and braided filaments, and/or another thread, filament, or strand-like construction. The textile strands 130 may be formed from filaments comprising one or more of a biocompatible metal of a permanent (e.g., stainless steel, titanium, etc.) or degradable nature (e.g., magnesium alloy, etc.); a biocompatible synthetic polymer of a permanent (e.g., polypropylene, polyester, etc.) or degradable nature (e.g., polylactic acid, polypropylene fumarate, polylactic-co-glycolic acid, etc.); and/or a collagen-based material (e.g., allograft, xenograft, etc.). In some embodiments, the mesh suture body 102 comprises a synthetic mesh, which is a mesh made from biocompatible and synthetic materials, such as polypropylene, polyethylene terephthalate polyester, expanded polytetrafluroethylene (ePTFE), polyglactin, polyglycolic acid, trimethylene carbonate, poly-4-hydroxybutyrate (P4HB), polyglycolide, polyactide, and/or trimethylene carbonate (TMC). In some embodiments, the body 52, 102 of the suture comprises a biological sheet or mesh, which is a sheet or mesh made from biocompatible and biological materials, such as human dermis, porcine dermis, porcine intestine, bovine dermis, and/or bovine pericardium. Additionally or alternatively, the body 52, 102 may be comprised of a combination of synthetic and biological materials and/or a combination of degradable and non-degradable materials, examples of which are described in more detail below.

In knitted, woven, and/or braided sutures, the textile strands 130 may be organized to optimize the biomechanical properties, such as tensile strength, as well as for porosity, morphology, and geometry as they relate to tensile strength and bioincorporation, which also influences tensile strength and frictional resistance. Various knitting techniques known in the art may be used to create the mesh suture body 102 of the suture 100. These include, but are not limited to, warp knitting, weft knitting, Crochet knitting, and Rachel knitting. Alternatively or additionally, various weaving techniques known in the art may be used to create the mesh suture body 102 of the suture 100. These include, but are not limited to, hexagonal open stitching (e.g., PARIETINE® mesh), interlocking fiber junctions (e.g., PROLENE® mesh, SURGIPRO Pro® mesh), diamond shape open stitching (e.g., ULTRAPRO® mesh), 2-dimensional weaves, and 3-dimensional weaves.

The mesh material forms a pattern of pores across the width W and length L of the porous suture 100, which may comprise one or several pores across the width and several pores along the length L. Similarly, the porous suture 50 may comprise any number of pores 70 across each of the width W and length L. The pores 70, 120 may be distributed in a consistent pattern across the length L and/or width W, or as described in more detail below, may be concentrated in certain areas along the length L or width W. The pores 70 in FIG. 1A are illustrated as substantially circular, and the pores 120 in FIG. 2A are substantially diamond-shaped. However, the pores 70, 120 may take on any shape opening formed by knitting, weaving, sewing, braiding, or otherwise linking textile strands or other materials that permits the passage of the suture through itself. In other embodiments, the pores 70, 120 may be round, oval, triangular, hexagonal, square, rectangular, or. In still other embodiments, the pores 70, 120 may be slits, or be elongated narrow openings, such that the pores are not easily visible when the body 52, 102 is laid flat but where the material on either side of the slits can be parted to reveal an opening.

With continued reference to FIGS. 2A-2C, the textile strands 130 of the mesh suture body 102 may collectively form an openwork structure or pattern that defines a plurality of pores 120, 122 arranged along the length L of the body 102 between the first and second ends 104, 106, and across the width W of the body 102 between the first and second lateral sides 108, 110. For example, as illustrated in FIGS. 1 and 2, the mesh suture 100 includes a plurality of central pores 120 arranged along the lateral midpoint of the body 102 between the first and second ends 104, 106, and a plurality of lateral pores 122 offset laterally relative to the central pores 120 towards the first or second lateral sides 108, 110.

As will be described in further detail below, the pores 70, 120, 122 are configured to allow the suture 50, 100 to be passed through itself when anchoring the suture 50, 100 to a target surface. Furthermore, the plurality of pores 70, 120, 122 give the suture body 52, 102 a lattice structure that may be useful, for example, to encourage tissue in-growth. As the tissue proximate to an implanted porous suture 50, 100 heals, scar tissue or other tissue types may form, grow, or otherwise extend into at least some of the pores 70, 120, 122. Engagement between the ingrown tissue and the pores 70, 120, 122 inhibits movement of the suture 100, thereby reducing movement of the suture 100 relative to the target surface once anchored thereto.

Similarly to the embodiment of FIG. 1, the body 102 of the mesh porous suture 100 may be configured to maintain a planar shape while the suture 50, 100 is under tension. This may be useful, for example, to increase the surface area of the suture 50, 100 on the target surface, thereby decreasing stress and distributing force in the target surface to reduce the risk of anchor point failure and preventing the pores 70, 120, 122 from deforming to a point that does not allow the ends 104, 106 of the suture 100 and/or the fixation device 114 to be passed through the pores 120 while the suture 100 is under tension. While the porous sutures 50, 100 of FIGS. 1 and 2 have bodies 52, 102 configured to maintain a planar shape, they are also sufficiently deformable so that they may be compressed, squeezed, folded, and/or rolled so that the sutures 50, 100 can be passed through the pores 70, 120.

While the embodiments shown are generally or substantially flat in thickness, in other embodiments the suture body 52, 102 may have a more substantial depth. While the porous sutures 50, 100 of FIGS. 1 and 2 are depicted as having generally planar bodies 52, 102 formed with a single layer, some embodiments may have multi-layer and/or a larger depth cross-section, such as a round, square, or triangular cross-section, to provide just a few examples. In such larger-depth embodiments, the suture body 52, 102 may have a solid or hollow cross-section. Optionally, such a larger depth suture may be configured such that it substantially flattens in thickness upon implantation, yet substantially maintains its width W.

In some embodiments, the suture 50, 100 is configured such that it maintains its width W, or a substantial portion of its width W, under lengthwise tension. For example, the suture 50, 100 is configured to remain flat and not arch, collapse, or otherwise change shape or width when a lengthwise force (i.e., in the direction along the length L between the first end and the second end) on the suture increases. This may be preferred for certain surgical applications to provide force distribution and adhesion, as described herein. In some embodiments, the material of the suture body 52, 102 is configured to maintain at least 50% of its width when under a lengthwise force of up to 16 N. In further embodiments, the material of the suture body 52, 102 Is configured to substantially maintain its width by maintaining at least 50% of its width when under a lengthwise force of up to 32 N/cm at the site of fixation with the tissue using 0.5 cm tissue bites (small bite standard). In further embodiments, the material of the suture body 52, 102 may be configured to substantially maintain its width by maintaining at least 60%, 70%, 80%, or 90%, or more, of its width when under a lengthwise force of up to 16 N or in some embodiments up to 32 N, or to some width value or force value in between. Conversely, the suture is configured such that its width decreases by no more than a given percentage under a predetermined lengthwise force, such as 16 N, 32 N, 50N, etc. when compared to the width W under no lengthwise force or compared to a minimal lengthwise force—e.g., a given percentage decrease of no more than 40%, 30%, 20%, or 10%. In still further embodiments, the material of the suture body 52, 102 may be configured to substantially maintain its width by maintaining at least 60%, 70%, 80%, or 90%, or more, of its width when under a lengthwise force of up to 50 N or more, or up to 200 N or more. For example, a group of porous sutures configured for Achilles tendon repair, such as via the Bunnell method, may be configured to collectively withstand a load of up to 200 N. For example, the force may be distributed over a group of four porous sutures 50, 100, where each porous suture is configured to withstand up to 50 N, or more, without attachment failure or suture failure. Other surgical applications may require withstanding loads of up to 100 N, or up to 200 N or more, without suture failure or attachment failure. The porous suture 50, 100 is dimensioned appropriately based on the load, such as having an appropriate width and structure to maintain sufficient width when under load.

The suture body 52, 102 may be formed of a material structure with low elongation along the axis of length L of the suture 50, 100, such as using a knit pattern configured to minimize such elongation. This minimizes the amount that the textile strands will collapse down when under lengthwise tension. For example, the mesh suture body 102 may be formed using a chain knit pattern in the machine direction (along the length direction of the textile strands). This minimizes the amount that the mesh will collapse down and deform along its length L when under lengthwise tension. Alternatively or additionally, the suture 50, 100 may be configured to have a greater width under lengthwise tension than its width W at rest, such as where the suture body 52, 102 is configured such that the depth (i.e., thickness dimension) collapses and the width W increases when a threshold amount of lengthwise tension is applied.

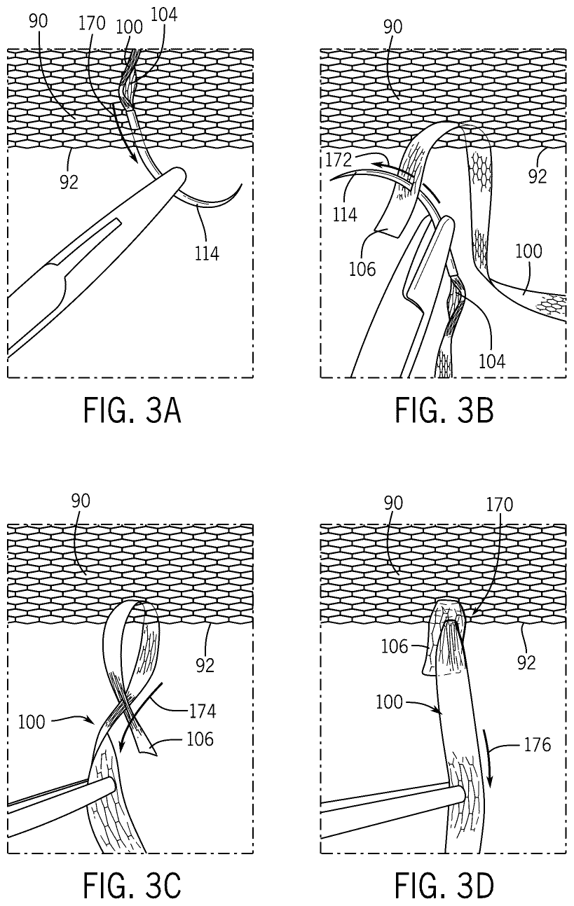

Thus, a plurality of strands running lengthwise in body may each be formed into chain stitches. The chain-stitched stands may then be connected across the width W by one or more cross-connecting strands, such as a set of weft strands woven, or otherwise knitted or linked into, the chain-stitched warp strands. The weave or knit pattern may be a loose pattern leaving spaces between the chain stitched longitudinal strands to form a mesh porous material where the mesh openings form the plurality of pores size to accommodate passing the porous suture, such as the porous suture 100 illustrated in FIGS. 2-3D. Alternatively, the weave or knit pattern may be a tighter formation leaving minimal spaces between the chain-stitched longitudinal strands to form a continuous sheet with minimal or no visible openings other than the plurality of pores being strategically formed to allow the porous suture to be passed through itself, such as the porous suture 50 illustrated in FIGS. 1A-1C. In one embodiment, the plurality of pores 70 may be formed by the weave or knit pattern so that the strands of the porous suture 50 in the continuous sheet are uncut. Alternatively, the plurality of pores 70 may be formed into the sheet by cutting holes in the fabric of the continuous sheet.

The material(s) selected for use in the porous suture 50, 100 of FIGS. 1A-1C and/or of FIGS. 2A-2C may be selected based on one or more desired parameters or characteristics of the porous suture 50, 100. For example, at least one material that forms a suture 50, 100 may be selected based on a desired modulus of elasticity and/or flexural rigidity so said the porous suture 50, 100 is flexible enough that it may be passed back and forth through target surfaces (e.g., tissue) and through its own body 52, 102 via the pores 70, 120. Additionally or alternatively, embodiments of the porous suture 50 of FIG. 1 and/or the suture 100 of FIG. 2 may be configured with a length dimension L, a width dimension W, and/or a thickness dimension T selected based on one or more desired parameter(s) of the porous suture 50, 100. For example, at least one of the length L, width W, and thickness T of the porous suture 50, 100 may be dimensioned based on the desired flexural rigidity of the porous suture 50, 100. In an exemplary embodiment, a porous suture 50, 100 may be configured to have a flexural rigidity that is less than or equal to 0.001 Pa*m\hat{ }4. Some embodiments, however, may be configured to have a flexural rigidity that is greater than 0.001 Pa*m\hat{ }4.

In some embodiments, the body 52, 102 of the porous suture 50, 100 may have a length L which permits multiple anchor points within the target surface upon implantation. An anchor point is a position where the suture 100 passes through some portion of the target surface to provide a force against migration or dehiscence. For example, as discussed in more detail below, each suture 50, 100 may be passed through the target surface multiple times, such as by weaving or sewing the porous suture 50, 100 into the tissue with at least one fixation device 114. Thereby, the porous suture 50, 100 may be configured such that it can withstand substantial forces, including for example, tensile stress, without failure. In an exemplary embodiment, the length L of a suture 50, 100 may be between 80 mm and 1000 mm long. Some embodiments, however, may have a length L that is shorter than 80 mm or longer than 1000 mm. For example, the length L of a suture 50, 100 may be 30 mm, 40 mm, 50 mm, 60 mm, 70 mm, 1100 mm, 1200 mm, 1300 mm, 1400 mm, 1500 mm, or any other length L suitable for the intended use of said suture 50, 100, including lengths L which are longer or shorter than the enumerated lengths L, and lengths L that are between any of the enumerated lengths L.

In some embodiments, the body 52, 102 of the porous suture 50, 100 may be dimensioned with a width W that is wide enough to distribute the force(s) acting on the target surface such that the porous suture 50, 100 does not cut or pull out of the target surface when acted upon by an internal or external force. In an exemplary embodiment, the width W of a suture 50, 100 may be between 2 mm and 15 mm wide. Some embodiments, however, may have a width W that is less than 2 mm or more than 15 mm. For example, the width W of a suture 50, 100 may be 0.5 mm, 1 mm, 1.5 mm, 20 mm, 40 mm, 60 mm, 80 mm, or any other width W suitable for the intended use of said suture 50, 100, including widths W that are more or less than the enumerated widths W, and widths W that are between any of the enumerated widths W.

In an exemplary embodiment, the thickness T of a suture 50, 100 may be between 0.1 mm and 2 mm. Some embodiments, however, may have a thickness T that is less than 0.1 mm or more than 2 mm. For example, the thickness T of a suture 50, 100 may be 0.02 mm, 0.04 mm, 0.06 mm, 0.08 mm, 4 mm, 6 mm, 8 mm, 10 mm, or any other thickness T suitable for the intended use of said suture 50, 100, including thickness T that is more or less than the enumerated thickness T, and thickness T that is between any of the enumerated thicknesses T.

In some embodiments, the length L of the suture 100 may be related to the width W of the suture 100. That is, the suture 100 may be configured with length and width dimensions selected based on a desired length-to-width aspect ratio (L:W) of the suture 100. Moreover, as described herein, the mesh suture 100 may be configured such that it does not deform under a lengthwise load, and thus may be configured such that the aspect ratio (L:W or W:L) does not substantially change (such as delimited by the percentage changes described above) when the porous suture is under high tension compared to the ratio under no lengthwise force. In an exemplary embodiment, a suture 50, 100 may be dimensioned to have a length-to-width aspect ratio that is at least 10 to 1 (10:1). For example, a suture 50, 100 may be 50 mm long and 5 mm wide (10:1), 100 mm long and 4 mm wide (25:1), 100 mm long and 2.5 mm wide (40:1), 1000 mm long and 15 mm wide (66.66:1), 1000 mm long and 2 mm wide (500:1), and/or any other combination of lengths and widths that results in an aspect ratio that is greater than 10 to 1, including aspect ratios that are between any of the enumerated aspect ratios. Some embodiments, however, may have an aspect ratio that is less than 10 to 1. For example, a suture 50, 100 may be 100 mm long and 15 mm wide (6.66:1), 50 mm long and 10 mm wide (5:1), 50 mm long and 15 mm wide (3.33:1), and/or any other combination of lengths and widths that results in an aspect ratio that is less than 10 to 1, including aspect ratios that are between any of the enumerated aspect ratios.

As previously mentioned, embodiments of a porous suture 50, 100 may be configured with pores 70, 120 that are dimensioned and/or arranged in the suture body 52, 102. The pores may be sized and shaped so that the suture 100 may be passed through itself via the pores 70, 120 in order to anchor the suture 50, 100 to the target surface. In some embodiments, the dimensions of the pores 70, 120 may be based on at least one of the dimensions of the fixation device(s) 114; the length L, width W, and/or thickness T of the suture body 52, 102; the properties of the material(s) that form the porous suture 50, 100; the properties of the target surface; the arrangement of pores 70, 120 in the suture body 52, 102; and any other parameter or characteristic of the porous suture 50, 100.

In an exemplary embodiment of a porous suture 50, 100, the pores 70, 120 may be dimensioned to have an effective diameter that is between 0.16 mm and 3.5 mm. For example, at least one pore 70, 120 may have a diameter of 1.75 mm, thereby giving the pores 70, 120 an open area of 2.5 mm². Some embodiments, however, may be configured with at least one pore 70, 120 with a diameter that is smaller than 0.16 mm or larger than 3.5 mm. For example, the diameter of at least one pore 70, 120 may be 0.05 mm, 0.1 mm, 4 mm, 4.5 mm, 5 mm, or any suitable diameter that is at least less than the width of the porous suture, including diameters that are more or less than the enumerated diameters, and diameters that are between any of the enumerated diameters.

In some embodiments, the spacing between the pores 70, 120 may be based on at least one of the dimensions of the fixation device(s) 114; the length L, width W, and/or thickness T of the suture body 52, 102; the diameter of the pores 70, 120; the properties of the material(s) that form the porous suture 50, 100; the properties of the target surface; and any other parameter or characteristic of the porous suture 50, 100. In an exemplary embodiment of a porous suture 50, 100, the pores 70, 120 may be spaced between 0.5 mm and 20 mm apart, center-to-center. For example, at least two adjacent pores 70, 120 may be spaced 5 mm apart. Some embodiments, however, may be configured with at least two adjacent pores 70, 120 that are less than 0.5 mm apart and/or more than 20 mm apart. For example, at least two adjacent pores 70, 120 may be 0.1 mm apart, 0.25 mm apart, 30 mm apart, 50 mm apart, 100 mm apart, and/or any other suitable distance, including distances that at are more or less than the enumerated distances, and distances that are between any of the enumerated distances.

Additionally or alternatively, the size, spacing, and/or arrangement of the pores 70, 120 may vary along the length L and/or width W of the suture body 52, 102. For example, an embodiment of a porous suture 50, 100 may be configured with pores 70, 120 that are more densely arranged at different locations on the porous suture 50, 100. The suture body 52, 102 may include a concentration of pores 70, 120 at locations where the porous suture 50, 100 will be passed through itself and a low density of pores 70, 120 at other locations where the porous suture 50, 100 will not be passed through itself. For example, a suture body 52, 102 may include a plurality of the pores 70, 120 proximate the first end 54, 104 and/or the second end 56, 106 and have zero or relatively few pores 70, 120 in a middle portion of the suture body 52, 102 compared to one or both of the end portions near the first end and/or the second end. This may be useful, for example, to provide a plurality of possible locations and options for passing the porous suture 50, 100 through itself near where an anchoring stitch may be formed or where increased tissue ingrowth is desired, while omitting pores 70, 120 at other locations where they are not needed, or where a stronger and/or more rigid length of suture may be desired or needed or less tissue ingrowth is desired. Alternatively, the suture body 52, 102 may include a concentration of pores 70, 120 some or all of the middle portion of the suture length L, such as for applications where the porous suture 50, 100 will be passed through itself to create anchoring points that are likely to align with that center section or to reduce foreign body material.

The pore size may be adapted for various embodiments and applications. In one embodiment, the pore size of the plurality of pores 70, 120 is consistent across the length L and/or width W of the suture body 52, 102. Alternatively, the pore size may vary along the length L of the suture body 52, 102. For example, the pores 70, 120 at or most proximate to the first end 54, 104 and/or the second end 56, 106 may be larger than the pores 70, 120 in the middle portion to enable initial anchoring of the proximal end of the porous suture 50, 100.

In addition or as an alternative to varying along the longitudinal length of a porous suture 50, 100, the pore density and/or pore size may vary across the lateral width of the suture body 52, 102 between the opposing lateral sides 58, 60, 108, 110 thereof. For example, along at least a portion of a porous suture 50, 100, the pores 70, 120 may be offset from the lateral midpoint of the suture body 52, 102 so that pores 70, 120 are included proximate one or both of the lateral sides 58, 60, 108, 110 and omitted or less densely arranged along the longitudinal centerline of the suture body 52, 102. In the case of a porous mesh suture 100, a portion of the suture body 102 may be configured with the central pores 120 omitted while the lateral pores 122 are still included. In some embodiments of a porous mesh suture 100, the density of the pores 120, 122 may be increased (and the size of the pores decreased) by more tightly weaving, sewing, and/or braiding the textile strands 130 and the density of the pores 120, 122 may be decreased (and the size of the pores increased) by more loosely weaving, sewing, and/or braiding the textile strands 130. The pores may be configured to constrict when the suture is put under tension thereby tightening around the porous suture where it passes through itself.

It should be appreciated that other lengths L, widths W, thicknesses T, aspect ratios, pore sizes, pore shapes, and/or materials are contemplated as within the scope of disclosure, and one of skill in the art will appreciate that various dimensions, materials and configurations may be appropriate depending on various parameters, such as the tissue defect or reconstruction and surgical approach by which the porous suture 50, 100 will be applied.

FIGS. 3A-3D, 4A-4D, and 5A-D illustrate exemplary methods of anchoring the porous sutures 50, 100 of FIGS. 1 and 2 to a target surface. While FIGS. 3A-3D, 4A-4D, and 5A-5D illustrate the use of a mesh suture 100 according to FIGS. 2A-2C, it should be appreciated that the illustrated procedures may also be used with a suture having a generally dense or tightly woven continuous sheet body, such as the porous suture 50 of FIGS. 1A-1C.

Referring to FIGS. 3A-3D, a porous suture 100 with a fixation device 114 at a first end 104 thereof may be passed through itself near an edge 92 of a target surface 90 to create a clinch knot 178 to anchor the suture 100 to the target surface 90. As illustrated in FIG. 3A, the fixation device 114 at the first end 104 is passed through the target surface 90 proximate to an edge 92 of said surface 90 in the direction of arrow 170. As illustrated in FIG. 3B, the fixation device 114 is then passed through a central pore 120 proximate to the second end 106 of the suture 100 in the direction of arrow 172. (Note: in FIG. 3B, the first end 104 of the suture 100 is obscured by the positioning instrument used to manipulate the suture 100). The suture 100 is then pulled by the first end 104 through the central pore 120 in the direction of arrow 174 to create a loop, as illustrated in FIG. 3C. The suture 100 is then pulled tight in the direction of arrow 176, as illustrated in FIG. 3D, to clinch the knot onto the target surface 90, thereby securing the suture 100 to the target surface 90 with a clinch knot 178 formed proximate to the second end 106 of the suture 100.

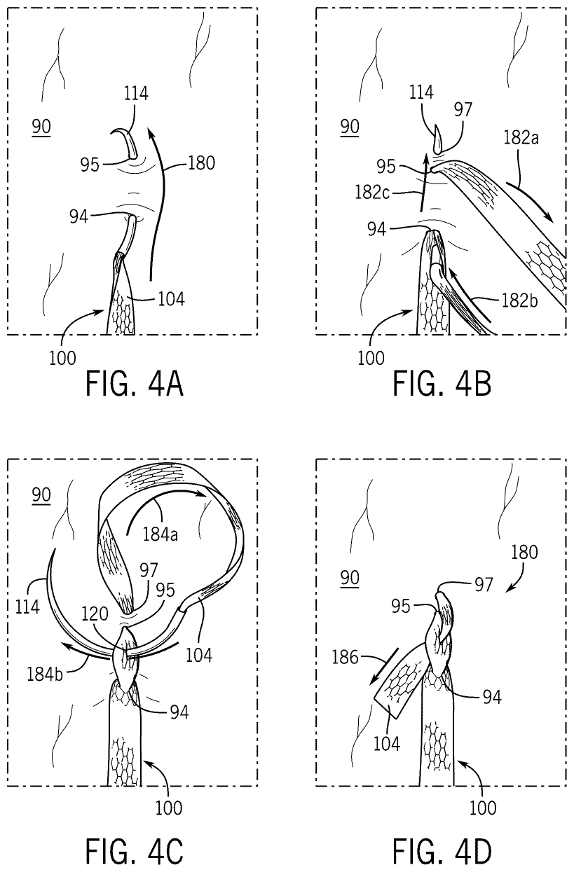

Referring to FIGS. 4A-4D, a porous suture 100 with a fixation device 114 at a first end 104 thereof may be passed through itself and a target surface 90, or alternatively to create a clinch knot across a wound, such as for tissue re-approximation. The locking stitch where the porous suture is passed through itself may be used alone as a stand-alone stitch or, for example, as a finishing anchor following a series of stitches for repairing tissue. As illustrated in FIG. 4A, the fixation device 114 at the first end 104 of the suture is passed into the target surface 90 at a first bite entry 94 and back out of the target surface 90 at a first bite exit 95 in the direction of arrow 180. The first end 104 of the suture 100 is then pulled out of the target surface 90 at the first bite exit 95 in the direction of arrow 182a and brought back around towards the first bite entry 94, as illustrated in FIG. 4B. The fixation device 144 is then passed through a central pore 120 of the suture 100 and back into the target surface 90 in the direction of arrow 182b via the first bite entry 94. The suture 100 is then pulled up through the target surface 90 in the direction of arrow 182c at a second bite exit 97 located just beyond the first bite exit 95. In the illustrated embodiments, the second bite exit 97 is located approximately 1 mm-2 mm beyond the first bite exit 95. In some embodiments, however, the second bite exit 97 may be located more than 2 mm beyond the first bite exit 95 or less than 1 mm beyond the first bite exit 95.

After the first end 104 has been pulled in the direction of arrow 184a through the second bite exit 97 to create a snug loop, the fixation device 114 is then passed in the direction of arrow 184b through a second centrally positioned pore 120 located between the first bite entry 94 and the first bite exit 95, as illustrated in FIG. 4C. Referring to FIG. 4D, the first end 104 of the suture 100 is drawn snug in the direction of arrow 186, thereby locking the loop in place to form the non-constricting self-locking backstitch 188. The suture 100 may then be cut proximate to the first end 104 at least 0.5 cm and preferably 1 cm or greater from the last point of exit 120 to remove the fixation device 114 and excess material from the suture 100.

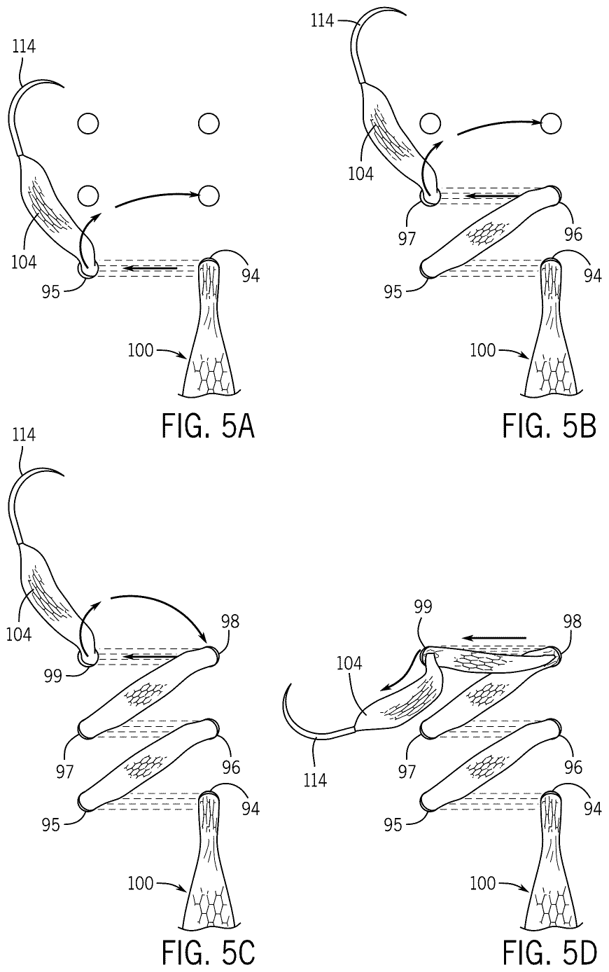

For example, the wound closure with the porous suture may be performed by securing the first end of the suture to tissue with a clinch knot, which is followed by a series of continuous stitches, and finished with a self-locking back stitch to anchor and secure the second end. FIGS. 5A-5D illustrate an exemplary self-locking stitch 190 for anchoring a mesh suture 100 with a fixation device 114 at a first end 104 thereof to a target surface 90, such as to repair tissue. As illustrated in FIG. 5A, the fixation device 114 at the first end 104 of the suture is passed into the target surface 90 at a first bite entry 94 and back out of the target surface 90 at a first bite exit 95 spaced laterally from the first bite entry 94, such as a first bite entry 94 on a first side of a tissue repair site and a first bite exit 95 on a second side of the tissue repair site. This process is repeated by passing the fixation device 114 into and out of the target surface 90 at a second bite entry 96 and a second bite exit 97 such as on the first and second sides to draw the porous suture across the tissue repair site multiple times to create a series of stitches crossing the tissue repair site, as illustrated in FIG. 5B. Then the implantation of the porous suture to repair tissue continues similarly, such as such as to close an incision or wound. Here, the fixation device 114 is passed into and out of the target surface 90 at a third bite entry 98 and a third bite exit 99 back on the first side of the tissue repair site, as illustrated in FIG. 5C. A locking stitch is then performed to finish off the series of stitches and anchor the first end 104 by passing the porous suture 100 through itself. Here, to create a finishing anchor, the first end 104 and the fixation device 114 attached thereto are passed through a central pore 120 of the suture 100 and back into the target surface 90 via the third bite entry 98. Further, as illustrated in FIG. 5D, the fixation device is then passed back out of the target surface 90 via the third bite exit 99 and through a second central pore 120 proximate the third bite exit 99. The same steps and method for creating a series of stitches and a finishing anchor could be performed with the continuous sheet porous suture 50 shown in FIGS. 1A-1C by passing the fixation device 114a through the appropriate pores 70.

Embodiments of a porous suture 50, 100 may additionally or alternatively be anchored to a target surface using other suturing methods or weave patterns. For example, a suture 50, 100 may be woven into the target surface in an x-weave pattern, a locking x-weave pattern, a plus weave pattern, a longitudinal weave pattern, a varied longitudinal weave pattern, or any other suturing weave pattern.

Passing the suture through itself, as exemplified in FIGS. 4A-5D and variously described herein, enables superior anchoring. Superior anchoring is enabled by the wider suture width that distributes force, and also from the ability to pass the suture through itself. Passing the suture through itself to create a self-locking stitch provides a flatter and less bulky knot with fewer layers of crossed material compared to traditional knotting techniques. The self-locking stitch lays relatively flat to the tissue, providing a less palpable knot that is less likely to cause tissue erosion. Moreover, the flatter knots enabled by the porous suture are less likely to harbor bacteria that cause infection and thus reduce instances of “stitch abscesses” or other infections that occur with larger knots having more interstices that harbor bacteria.

In some embodiments, the body 52, 102 of a suture 50, 100 and/or the textile strands 130 that make up the mesh suture body 102, or the suture body 102 may be coated in part or in total to enhance at least one of tensile strength, frictional resistance, lubricity, anti-adhesion, tissue response, and bioincorporation. Additionally or alternatively, the body 52, 102 of a suture 50, 100 and/or the textile strands 130 that make up the mesh suture body 102 may be treated with a medicament coating configured to deliver a medication to the target surface at the site of the porous suture 50, 100.

Some embodiments of a mesh porous suture 100 may be formed with more than one type of textile strands 130. In some embodiments, such as the embodiment of FIGS. 2A-2C, the textile strands 130 forming the mesh suture body 102 of the suture 100 may have smooth sides to reduce the friction and/or other forces that resist the movement of the suture 100 through and on the target surface. However, some embodiments of a mesh porous suture 100 may include at least one barbed strand 132 with rough edges and/or barbs that are configured to engage the target surface and/or another portion of the suture body 102 to increase the force required to draw the suture 100 through the target surface and/or through the pores 120 in the body 102. This may be useful, for example, to hold tension on the mesh suture 100 as it is inserted into the target surface. The rough surface and/or barbs on each barbed strand 132 may be configured to equally resist movement of the strand 132 in both directions, or they may be configured to create more resistance in one direction relative to the opposite such that the suture 100 is easier to pull in one direction through tissue and/or itself than in the opposing direction. Alternatively, the rough surface and/or barbs may protrude from the mesh suture body when loaded in tension.

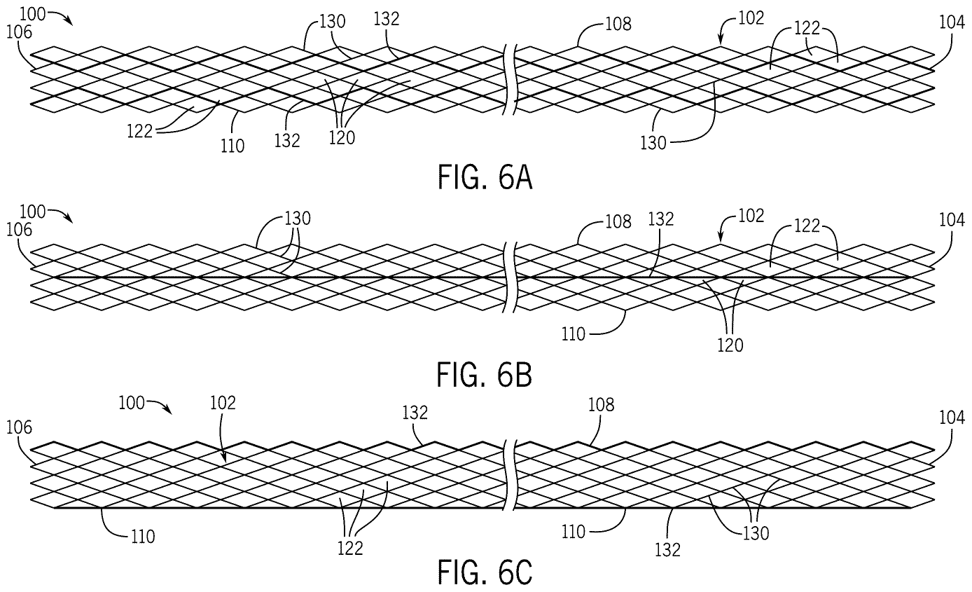

FIGS. 6A-6C illustrate embodiments of a mesh suture 100 that include at least one barbed strand 132, which may be a strand including one or more barbed filaments. Each barbed strand 132 may be interwoven with the non-barbed textile strands 130 to form the mesh suture body 102. For example, FIG. 6A illustrates an embodiment of a mesh suture 100 that includes two barbed strands 132 incorporated into the mesh suture body 102. Barbs may be created by, but not limited to: knitting a mesh with a portion of the fibers projecting from one or both surfaces of the mesh suture body, where in some embodiments the projecting yarn is cut to create the barbs; or the mesh suture body is knit in a double-face construction and the connecting fibers cut to create two separate one-sided barbed mesh sutures; or loose fibers incorporated into the mesh suture body during the knitting process. Additionally or alternatively, barbs or barb-like texture can be produced after knitting/forming to affect one or both sides in a specific orientation through post processing cutting, spraying, etc. For example, barbs may be molded into the strands, or welded/glued onto either the induvial strands or the mesh suture body, or may be created by “back-cutting” the strands leaving knicks, or the edge of the mesh suture body cut roughly exposing barb like features.

Some embodiments of a mesh suture 100 may include at least one barbed strand 132 that is integrated into a mesh suture body 102 formed from non-barber textile strands 130. For example, FIG. 6B illustrates an embodiment of a mesh suture 100 that includes one barbed strand 132 that extends longitudinally along the length of the suture 100. In FIG. 6B, the illustrated barbed strand 132 extends along the lateral center of the suture body 102 and extends through the central pores 120. Some embodiments, however, may include a barbed strand 132 that is offset from the lateral center of the suture body.

Some embodiments of a mesh porous suture 100 may include at least one barbed filament extending along one of the lateral edges 108, 110 of the suture body 102. For example, FIG. 6C illustrates an embodiment of a mesh suture 100 that includes barbed filaments extending along the first lateral side 108 and the second lateral side 110 of the suture body 102. In FIG. 6C, the barbed strand 132 extending along the first lateral side 108 is interwoven with the other textile strands 130 to form the mesh suture body 102 while the barbed strand 132 extending along the second lateral side 110 is integrated into the non-barbed textile strands 130 and extends linearly between the first and second ends 104, 106 of the suture body 102.

Additionally or alternatively, some embodiments of a porous suture 50, 100 may include at least one biodegradable filament and/or textile strand 130 configured to degrade over time after said biodegradable filament and/or textile strand 130 is inserted into tissue or another target surface. In such an embodiment, the biodegradable filaments may hold tissue together while it heals before degrading when the additional filament is no longer needed. For example, a porous suture 50, 100 may include biodegradable barbed strands 132 or at least some biodegradable filaments that are configured to hold tension in the suture 100 as it is inserted before degrading after insertion. In such an embodiment, the barbed suture(s) strands 132 may be configured to degrade entirely, or the barbs on the barbed strands 132 or barb filaments may degrade while the body of the strand 132 remains.

Embodiments of a porous suture 50, 100 may include different quantities, arrangements, and combinations of biodegradable filaments and/or textile strands 130. For example, some embodiments may be configured with between 25% and 75% biodegradable filaments and/or textile strands 130. Other embodiments, however, may be configured with less than 25% or more than 75% biodegradable filaments and/or textile strands 130. In some embodiments, at least one textile strand 130 may include some biodegradable filaments and some non-biodegradable filaments. For example, a textile strand 130 may include 50% biodegradable filaments and 50% non-non-biodegradable filaments. Other textile strands 130 may be configured with less than 50% biodegradable filaments or more than 50% biodegradable filaments.

Some embodiments of a porous suture 50, 100 may include biodegradable filaments and/or textile strands 130 at select locations on the suture body 102 while non-biodegradable filaments and/or textile strands 130 are included at other locations on the suture body 102. For example, a porous mesh suture 100 may include at least one section of biodegradable strands or filaments that extend longitudinally across the mesh suture body 102 and/or at least one section of biodegradable filaments that extend laterally across the mesh suture body 102. In such an embodiment, the biodegradable filaments may be arranged in a pattern that results in a desired shape and/or size of the suture body 102 once the biodegradable filaments degrade and the non-biodegradable filaments remain. The same may also be true for the continuous sheet porous suture embodiment 50, where the mesh body 52 may comprise degradable strands or filaments comprising some or all of the material thereof. In an embodiment incorporating both biodegradable materials or both biodegradable and nonbiodegradable/permanent strands or filaments, there may be a decrease in longitudinal stiffness over time as the tissue heals and the biodegradable strands break down over time.

Some embodiments of a porous suture 50, 100 may include an encapsulating material (not shown) that is wrapped around, formed on, or otherwise covers around a portion of the suture body 52, 102. The encapsulating material may be configured to change the cross-sectional shape of the suture body 52, 102 when applied thereto and/or it may provide an outer surface that has different properties than that of the uncovered porous suture 50, 100. This may be useful to assist the user in inserting or passing the porous suture 50, 100 into or through the target surface or anchoring the porous suture 50, 100 to the target surface.

For example, embodiments of a porous suture 50, 100 may include an encapsulating material that encloses a portion of the first end 54, 104 or the second end 56, 106 of the body 52, 102 and/or any fixation device 114 attached thereto. The encapsulating material may be configured to compress the covered portion of the porous suture 50, 100 to deform the suture body 52, 102 from its planar shape to have a smaller, narrower, or more circular cross-section with a smooth outer surface. This may be useful, for example, so that the porous suture 50, 100 can be more easily passed through the target surface and/or the pores 70, 120. Additionally or alternatively, the encapsulating material may have a surface texture that is more easily grasped by the user's hand or surgical instrument. In some embodiments, the encapsulating material may enclose one or each of the first or second end 54, 56, 104, 106 to create a rigid or semi-rigid end structure on the suture that can be used to facilitate fixation. For example, the encapsulated end may function similar to a needle, and may take a curved, straight, or S-shape. In other embodiments, the encapsulating material may enclose one or each of the first or second end 54, 56, 104, 106 may enable attachment to another fixation device 114, such as a needle, by providing a narrower and/or stiffer attachment location. In other embodiments, the encapsulating material may enclose one or each of the first or second end 54, 56, 104, 106 and a portion of the attached fixation device 114 (such as a needle), so that the encapsulation material covers the hub of the fixation device 114 at the connection between the fixation device 114 and the suture body 52, 102.

Embodiments of the encapsulating material may take a variety of different forms that can be applied to the suture body 52, 102. For example, the encapsulating material may be configured as an elongated strip of material that is wrapped around the suture body 52, 102; a material that is disposed on the suture body 52, 102 in a liquid or semi-liquid form before solidifying; a sheath with a tubular body that can slide onto the suture body 52, 102; a deformable material that is crimped around the suture body 52, 102; thin thread whipped around the end of the suture body 52, 102; and/or any other material type or arrangement that can be used to enclose a portion of the suture body 52, 102. In embodiments where the encapsulating material is configured as a sheath, the sheath can slide onto the suture body 52, 102 by passing the first or second end 54, 56, 104, 106 and/or any attached fixation device 114 through the tubular sheath. In some embodiments, the sheath may be configured as a heat-sensitive shrink wrap that is configured to contract when exposed to a heat source, such as comprised of a medical-grade PET or FEP heat shrink tubing. The shrink wrap may have an initial diameter that allows it to easily slide onto the suture body 52, 102. After the shrink wrap is in the desired position on the porous suture 50, 100, heat may be applied to the sheath, causing the shrink wrap to contract on the suture body 52, 102 to encapsulate the body 52, 102 and compress the body 52, 102 into a circular cross-section.

In some embodiments, a fixation device may be formed on the suture body 52, 102 by applying an encapsulating material to the first end 54, 104 and/or the second end 56, 106 of the porous suture 50, 100. For example, an encapsulating material configured to compress the suture body 52, 102 may be applied to the first end 54, 104 and/or the second end 56, 106 thereof. The encapsulating material will compress the end 54, 56, 104, 106, causing the suture body 52, 102 to taper from a planar cross-section to a streamlined cross-section that can easily penetrate the target surface and/or passed through the suture body 52, 102. For example, a shrink wrap sheath may be positioned on the suture body 52, 102 such that a portion of the sheath extends past the first or second end 54, 56, 104, 106 of the suture body 52, 102. When heat is applied to the shrink wrap, the portion of the shrink wrap extending past the end 54, 56, 104, 106 of the suture body 52, 102 may deform into a generally pointed shape that can penetrate the target surface. The encapsulating material may be used to form a fixation device with a specific shape that allows the porous suture 50, 100 to be passed through the target surface and the suture body 52, 102 in a particular manner according to a desired anchoring stitch pattern. For example, an encapsulating material may be used to form a fixation device that is straight, curved, S-shaped, and/or any other desired form on the first end 54, 104 and/or the second end 56, 106 of the porous suture 50, 100.

Additionally or alternatively, some embodiments of a porous suture 50, 100 may be configured with a first end 54, 104 and/or a second end 56, 106 that can be formed into a fixation device without additional encapsulating material. For example, in an embodiment of a porous mesh suture 100, the textile strands 130 may be melted by exposure to heat or chemical application enabling that exposed portion of the material to be deformed into a streamlined shape to form a rigid or semi-rigid fixation device or attachment point for a fixation device. As with a fixation device formed with an encapsulating material, the ends 54, 56, 104, 106 of the suture body 52, 102 may be modified to form a fixation device having a desired shape, including a straight fixation device, a curved fixation device, a S-shaped fixation device, and/or a fixation device having any other desired shape.

### EXAMPLES

The following examples, test embodiments, test setups, and test data, are meant only to be illustrative and are not meant as limitations on the scope of the invention or of the claims.

EXAMPLE A: The porous suture was tested against a standard-of-care #0 propylene suture, and particularly to test the comparative mechanical strength of the porous suture compared to the standard-of-care (SOC) suture when used to form a locking stitch for anchoring to tissue. For the porous suture, a mesh porous suture comprising a 6 mm wide polypropylene mesh formed of a Crochet warp knit pattern was utilized. A respective end of each of the mesh porous suture and the SOC suture were anchored to a slab of pig abdominal wall tissue using an affixed GS21 needle. One end of the mesh porous suture was anchored to the tissue using a locking backstitch disclosed herein wherein the porous suture was passed through itself, such as illustrated and described with respect to FIGS. 4A-4D. One end of the SOC suture was anchored to the tissue using a standard surgeon's knot followed by four throws.

The maximum load capacity of the anchoring was then tested using an Instron Model 1321 test system through vertical distraction. The free end of the porous suture and the SOC suture were connected to the Instron, as shown below, and tensile testing was adapted from USP881 (Tensile Strength of Surgical Suture). The vertical displacement was increased at a rate of 16 cm/min and load recorded at a rate of 100 Hz until failure occurred, which included any one of i) suture failure where the suture completely tore or otherwise broke; ii) tissue failure where the suture remained attached to a portion of the tissue that detached from the remainder of the tissue; iii) fixation failure where the suture knot or anchor point unraveled or otherwise failed such that the suture slid through and out of the tissue.

The maximum load of the anchor for each of the SOC suture and the mesh porous suture was determined. The results demonstrate that the porous suture had a significantly higher maximum load than the SOC suture. The porous suture anchor that utilized the disclosed locking backstitch was able to withstand over twice the max load capacity compared to the SOC suture anchored by a standard surgeon's knot, which in the test results exceeded 90 N.

EXAMPLE B: Various porous sutures having widths between 2 mm and 6 mm were created and tested to compare mechanical strength and stiffness of mesh porous sutures of varied widths, including mesh porous sutures having widths of 2 mm, 4 mm, and 6 mm. Each porous suture was created from a 10 mm wide section of polypropylene mesh formed of a Crochet warp knit pattern, cutting 10 mm wide pieces of mesh into one piece having a width of 2 mm, a second piece having a width of 4 mm, and a third piece having a width of 6 mm.

Each of the three mesh widths (N=5) representing porous sutures of the above-mentioned widths was placed in an Instron Model 1321 test system to test vertical distraction per ASTM D5035 (Breaking Force and Elongation of Textile Fabrics), and thereby to test the maximum load of each piece. The vertical displacement was increased at a rate of 100 mm/min, starting at 2 N preload, until complete failure (tear) of the mesh piece.

The failure load of each mesh porous suture was determined. A longitudinal stiffness value (load/displacement) was also determined for each mesh porous suture. The results demonstrate that, for the dimensions tested, increasing the width of the porous suture increases the maximum load of the porous suture and increases the longitudinal stiffness of the porous suture. (See Table A)

TABLE A
A table of the maximum “failure” load and the longitudinal stiffness
for each of the 2 mm, 4 mm, and 6 mm porous suture widths.

| Mesh | Failure | Stiffness |
|---|---|---|
| Width | Load (N) | (N/mm) |
| 2 mm | 41.45 ± 2.08 | 2.37 ± 0.07 |
| 4 mm | 81.74 ± 13.37 | 4.14 ± 0.33 |
| 6 mm | 104.14 ± 6.96 | 5.80 ± 0.18 |

This written description uses examples to disclose the invention and to enable any person skilled in the art to make and use the invention. Certain terms have been used for brevity, clarity, and understanding. No unnecessary limitations are to be inferred therefrom beyond the requirement of the prior art because such terms are used for descriptive purposes only and are intended to be broadly construed. The patentable scope of the invention is defined by the claims and may include other examples that occur to those skilled in the art. Such other examples are intended to be within the scope of the claims if they have features or structural elements that do not differ from the literal language of the claims, or if they include equivalent features or structural elements with insubstantial differences from the literal languages of the claims.

What is claimed is:

1. A porous suture for tissue repair, the porous suture comprising:
a planar body having a length between a first end and a second end and a width between a first lateral side and a second lateral side;
the planar body formed of a porous material comprising a plurality of pores along the length of the planar body between the first end and the second end; and
wherein the planar body is configured to substantially maintain the width despite an increase in a lengthwise force on the planar body.

2. The porous suture of claim 1, wherein the width of the suture decreases by no more than 40% when the lengthwise force increases up to 16N compared to the width under no lengthwise force.

3. The porous suture of claim 1, wherein the width of the suture decreases by no more than 40% when the lengthwise force increases up to 32N compared to the width under no lengthwise force.

4. The porous suture of claim 1, wherein the width of the suture decreases by no more than 40% when the lengthwise force increases up to 50N compared to the width under no lengthwise force.

5. The porous suture of claim 1, wherein the width of the suture decreases by no more than 30% when the lengthwise force increases up to 16N compared to the width under no lengthwise force.

6. The porous suture of claim 1, wherein the width of the suture decreases by no more than 10% when the lengthwise force increases up to 16N compared to the width under no lengthwise force.

7. The porous suture of claim 1, wherein the planar body is configured to remain flat along its width despite the increase in lengthwise force.

8. The porous suture of claim 1, wherein the planar body comprises a plurality of strands spread across the width between the first lateral side and the second lateral side, wherein the plurality of strands form chain stitches along the length.

9. The porous suture of claim 8, wherein the porous material is a mesh and further comprises a set of cross-connecting strands connecting the chain stitches to form the mesh.

10. The porous suture of claim 9, wherein the mesh forms the plurality of pores, and wherein a pore size of the plurality of pores is consistent along the length of the planar body.

11. The porous suture of claim 9, wherein the mesh forms the plurality of pores, and wherein a pore size of the plurality of pores is larger near the first end and/or the second end than in a middle portion of the planar body.

12. The porous suture of claim 9, wherein the mesh forms the plurality of pores, and wherein a pore size of the plurality of pores is larger near a longitudinal centerline of the planar body than at the first lateral side or the second lateral side.

13. The porous suture of claim 1, wherein the porous material is a continuous sheet, wherein the plurality of pores are formed through the sheet.

14. The porous suture of claim 1, wherein the plurality of pores are aligned along a longitudinal centerline of the planar body.

15. The porous suture of claim 1, wherein at least a portion of the plurality of pores is sized to accommodate the porous suture being passed there through, such that the porous suture is configured to be passed through itself.

16. The porous suture of claim 1, wherein the plurality of pores are uniformly spaced along the length of the planar body between the first end and the second end.

17. The porous suture of claim 1, wherein the width is at least 4 mm and a ratio of the width to the length of the planar body is at least 1:10, and wherein the ratio does not substantially change when the porous suture is under high tension compared to the ratio under no lengthwise force.

18. The porous suture of claim 1, wherein the planar body comprises a single ply of the porous material.

19. The porous suture of claim 1, further comprising a first surgical needle fixed to the first end of the planar body and/or a second surgical needle fixed to the second end of the planar body, wherein the planar body is configured to substantially maintain the width despite the increase in the lengthwise force on the planar body applied from the first surgical needle and/or the second surgical needle.

20. A method of repairing tissue with a porous suture, the porous suture comprising a planar body having a length between a first end and a second end and a width between a first lateral side and a second lateral side, with a plurality of pores formed along the length of the planar body between the first end and the second end, the method comprising:
passing the first end of the porous suture through tissue on each of a first side of a tissue repair site and a second side of the tissue repair site to create at least one stitch; and
passing the suture through itself to create a self-locking stitch to create a finishing anchor at the first end of the porous suture.

21. The method of claim 20, further comprising passing the first end of the porous suture through tissue on each of the first side and the second side of the tissue repair site multiple times to create a series of stitches to close a wound, wherein the self-locking stitch follows the series of stitches.
